(19) United States
(12) Patent Application Publication
(10) Pub. No.: US 2019/0314045 A1
(43) Pub. Date: Oct. 17, 2019

(54) TARGETING METHODS AND DEVICES FOR NON-INVASIVE THERAPY DELIVERY

(71) Applicants: Bryan Cunitz, Seattle, WA (US); Kennedy Hall, Seattle, WA (US); Mathew Sorenson, Seattle, WA (US); Michael R. Bailey, Seattle, WA (US); Adam D. Maxwell, Seattle, WA (US); Barbrina Dunmire, Seattle, WA (US); Oren Levy, South San Francisco, CA (US); Doug Corl, South San Francisco, CA (US); Paul Fasolo, South San Francisco, CA (US)

(72) Inventors: Bryan Cunitz, Seattle, WA (US); Kennedy Hall, Seattle, WA (US); Mathew Sorenson, Seattle, WA (US); Michael R. Bailey, Seattle, WA (US); Adam D. Maxwell, Seattle, WA (US); Barbrina Dunmire, Seattle, WA (US); Oren Levy, South San Francisco, CA (US); Doug Corl, South San Francisco, CA (US); Paul Fasolo, South San Francisco, CA (US)

(21) Appl. No.: 16/383,386

(22) Filed: Apr. 12, 2019

Related U.S. Application Data
(60) Provisional application No. 62/656,869, filed on Apr. 12, 2018.

(51) Int. Cl. A61B 17/22; A61B 8/08; A61B 5/00; A61B 8/00

(52) U.S. Cl. CPC A61B 17/22004; A61B 8/085; A61B 5/7405; A61B 2017/00106; A61B 8/462; A61B 8/488; A61B 8/5246; A61B 5/7455

(57) ABSTRACT

Targeting methods and devices for non-invasive therapy delivery are disclosed. In one embodiment, a method for targeting an object in a body using ultrasound includes: producing a therapy ultrasound waveform configured to fragment or comminute the object in the body using a therapy transducer of an ultrasound probe; and acquiring a sound waveform by a receiver. The sound waveform is at least in part caused by interactions of the therapy ultrasound with the object. The method also includes generating an indication of a targeting accuracy based on the acquired sound waveform.

### CROSS-REFERENCE TO RELATED APPLICATION

This application claims the benefit of Provisional Application No. 62/656,869, filed Apr. 12, 2018, which is incorporated herein by reference.

### BACKGROUND

Ultrasound has been employed to diagnose and facilitate a non-invasive removal of soft tissues such as tumors or calcifications such as kidney stones from the body.

Ultrasound can also be used to noninvasively image stones or other objects in the body, manipulate and move these objects, or fragment them into small pieces so that they can be removed more easily.

FIG. 1 is a partially schematic view of an ultrasound system 100 in accordance with conventional technology. The ultrasound system 100 includes a therapy probe 14 and an imaging probe 22. The therapy probe 14 can incorporate one or more piezoelectric transducer elements 14i that expand and contract with the changing polarity of electrical voltage applied to the transducer. Such a change in polarity can be generated at a target ultrasound frequency by an alternating current (AC) generator 12. In operation, the therapy transducer 14i vibrates at a prescribed frequency of the therapy ultrasound (corresponding to the AC frequency) when activated by a therapy trigger module 10. These vibrations generate ultrasound waves that propagate through the body and toward a target object (e.g., a kidney stone). The ultrasound may be focused onto the target object by a lens attached to the therapy transducer 14i or by the shaped surface of the therapy transducer itself.

The illustrated ultrasound system 100 includes an imaging probe 22 incorporating one or more piezoelectric transducer elements 22i. The operation of the imaging probe 22 is analogous to that of the therapy probe 14. Namely, the imaging transducer elements 22i also transmit ultrasound waves toward the object of interest, but typically at a frequency that is different from that of the therapy transducer. When the imaging ultrasound waves impinge on the object of interest (e.g., a kidney stone) and reflect back toward the piezoelectric elements 22i of the imaging probe 22, the reflected ultrasound waves generate AC signals in the imaging transducer elements 22i. These AC signals are processed by an imaging system 20, and displayed on a display 30 of the system to provide an indication of, for example, shape, location, or motion of the object of interest or the surrounding tissue.

In some systems, the therapy probe and imaging probe are coupled to form a combined probe. An example of such combined probe is illustrated in FIG. 2 where an ultrasound probe 110 includes the therapy probe 14 and the imaging probe 22. The therapy probe 14 and the imaging probe 22 may take turns when operating to reduce interference between the therapy and imaging ultrasound.

FIG. 3 is an isometric view of a combined ultrasound probe in operation in accordance with conventional technology. In use, an operator 40 holds a handle 16 to aim the combined ultrasound probe 110 toward the target object in a patient 50. This targeting is based on the information obtained by the imaging probe 22. The operator 40 typically adjusts his/her aim by adjusting the position and/or angle to the ultrasound transducer 110 from time to time.

However, due to limitations of ultrasound imaging, patient motion, and other system limitations, there may be a loss of targeting accuracy, wherein the therapy probe 14 is not optimally aimed towards the therapy target (e.g., kidney stone). Furthermore, the images obtained by the imaging transducer 22 typically correspond to a single focal plane in a 3D space, therefore providing limited feedback regarding the displacement of the target object out of the plane of the image. For more comprehensive imaging of the target object, multiple focal planes may be helpful. However this added feature may reduce the imaging frame rate or otherwise compromise the image quality, while greatly increasing the complexity of the imaging probe and imaging system. Accordingly, there remains a need for improved targeting systems and methods for the therapy ultrasound.

### SUMMARY

This summary is provided to introduce a selection of concepts in a simplified form that are further described below in the Detailed Description. This summary is not intended to identify key features of the claimed subject matter.

Briefly, the inventive technology is directed to systems and methods for locating and targeting objects in a body. With conventional technologies, an operator often does not have good visualization of the desired target. While diagnostic ultrasound imaging of the conventional technology can provide image guidance and visualization of an in-vivo target, it cannot always indicate whether the therapy pulse is targeting the target object correctly or whether the therapy ultrasound is having the desired therapeutic effect.

The inventive technology improves targeting of the objects by therapeutic ultrasound. In some embodiments, targeting is based on the therapy ultrasound that is reflected toward ultrasound receivers. Linking the emitted therapy ultrasound to the received reflected therapy ultrasound may provide an indication of the accuracy of the targeting (also referred to as the “accuracy of locating” or “accuracy of aiming”). Different embodiments of the inventive technology may be used to treat kidney stones, gallbladder stones, arterial calcification, cardiac valve calcification, and/or other target objects. When the emitted therapeutic ultrasound impinges upon a hard object (e.g., a stone or a calcification) in the body, the reflected therapy ultrasound may have higher amplitudes or specific frequencies which are not present in the therapy ultrasound that reflected from a soft tissue, which is then used to confirm the accuracy of the targeting.

In some embodiments, targeting is based on the sound emissions associated with the therapeutic activity arising from the interaction of the therapeutic ultrasound waves impinging on the therapy target (e.g., kidney stone). In particular, the nature of the sound waveform is indicative of the effectiveness of the therapy, and by inference, how well the therapy ultrasound is targeted on the object. In such embodiments, the sound emissions may be acquired by audio microphones, since the therapy ultrasound, which was outside of the audible range, creates audible sounds through the therapeutic interaction with the target object. Such therapeutic interactions may arise from cavitation bubble collapse, stone fragmentation, stone vibration, stone movement, or other nonlinear interactions between the therapeutic ultrasound and the target object. These nonlinear therapeutic interactions ultimately cause an audible component in the sound, where this audible component, when properly captured and interpreted, provides an insight into location, shape, size, movement, and/or other properties of the target.

In some embodiments, the audible sound may be acquired by the elements of the therapy probe itself (e.g., by the piezo elements of the phased array therapy transducer acting as sound receivers). In different embodiments, the inventive technology may be practiced in conjunction or in absence of the conventional imaging transducers. The technology disclosed herein can be based on different types of therapy ultrasound, including Burst Wave Lithotripsy (BWL), Shockwave Lithotripsy (SWL), or histotripsy. When the therapy ultrasound is emitted in bursts of ultrasound waves (BWL), the bursts are separated by the rest periods when the ultrasound is not emitted. Therefore, the reflected signal may also encode frequency components related to the repetition frequency of the bursts, that frequency being generally lower and, therefore, closer to or within the audible frequency range than the frequency of the ultrasound waves within individual bursts.

In some embodiments, the sound emissions associated-with and caused-by the therapeutic ultrasound impinging on the therapy target (e.g., kidney stone) are processed to provide indicators of the target accuracy that guides the operator during the therapy. Some examples of such indicators of the target accuracy are arrows, target icons, sound indicators emitted by speakers, and haptic indicators. In some embodiments, a robotic manipulator can change the orientation and/or focal plane of the therapy ultrasound transducer based on the accuracy indicators.

In one embodiment, a method for targeting an object in a body using ultrasound includes: producing a therapy ultrasound waveform configured to fragment or comminute the object in the body using a therapy transducer of an ultrasound probe; acquiring a sound waveform by a receiver, where the sound waveform is at least in part caused by interactions of the therapy ultrasound with the object; and generating an indication of a targeting accuracy based on the acquired sound waveform.

In one embodiment, the object is a stone or a calcification.

In one embodiment, the receiver comprises a microphone.

In another embodiment, the microphone and the therapy transducer are carried by a common housing of the ultrasound probe.

In one embodiment, the microphone and the therapy transducer are separate.

In one embodiment, the method also includes: converting a microphone signal into a digitized signal in a time domain; processing the digitized signal into a frequency spectrum; detecting at least one extremum in the frequency spectrum; and determining the targeting accuracy based on the at least one extremum of the frequency spectrum. In one embodiment, generating the indication of the targeting accuracy includes generating an audible feedback or a light feedback.

In another embodiment, generating the indication of the targeting accuracy includes generating a haptic feedback.

In one embodiment, generating the indication of the targeting accuracy includes generating an image on a display unit, where a shape, a size or a color of the image indicates the targeting accuracy.

In one embodiment, the method also includes: retargeting the therapy ultrasound waveform based on the indication of the targeting accuracy.

In one embodiment, retargeting the therapy ultrasound waveform includes robotically retargeting the therapy ultrasound waveform.

In one embodiment, the therapy probe incorporates a phased array therapy transducer having a plurality of individually operable transducer elements. The method further includes retargeting the therapy ultrasound waveform by controlling the electrical excitations of the individual elements of the phased array.

In one embodiment, the sound waveform includes sound emissions from cavitation bubbles generated by the therapy ultrasound waveform.

In one embodiment, the therapy ultrasound waveform is transmitted in bursts, where a frequency of the bursts is within an audible range of frequencies, and the sound waveform includes the frequency of the bursts.

In one embodiment, the method includes: generating a Doppler ultrasound audio waveform using an imaging transducer of the ultrasound probe; and generating a display representative of the object motion based on the imaging ultrasound waveform.

In one embodiment, the imaging ultrasound waveform includes a pulse wave Doppler (PWD) ultrasound.

In one embodiment, an apparatus for treating an object in a body using ultrasound includes: an ultrasound probe having a therapy transducer configured to fragment or comminute the object in the body by a therapy ultrasound, and an imaging probe configured to image the object by an imaging ultrasound. The receiver is configured to detect a sound waveform, where the sound waveform is at least in part caused by interactions of the therapy ultrasound with the object. The apparatus also includes an indicator configured to indicate a targeting accuracy based on the sound waveform detected by the receiver.

In one embodiment, an apparatus for treating an object in a body using ultrasound includes an ultrasound probe having a therapy transducer configured to fragment or comminute the object in the body by a therapy ultrasound, and an imaging probe configured to image the object by an imaging ultrasound. The apparatus also includes a receiver configured to detect a sound waveform; and an indicator configured to indicate a targeting accuracy based on the reflected ultrasound waveform detected by the receiver.

In one embodiment, the receiver is a microphone.

In one embodiment, the apparatus further includes a controller configured to adjust a target therapy zone based on the targeting accuracy.

In another embodiment, the apparatus further includes a robotic arm configured to adjust a position of the ultrasound probe.

In one embodiment, the therapy transducer is a phased array therapy transducer comprising a plurality of individually operable transducer elements that are configured to generate the therapy ultrasound and to detect the sound waveform.

In one embodiment, the receiver is a bed-side microphone.

In one embodiment, the receiver is a microphone, and the microphone and the therapy transducer are carried by a common housing of the ultrasound probe.

In one embodiment, the apparatus also includes at least one of a speaker or a source of light operationally coupled with the indicator configured to indicate the targeting accuracy.

In one embodiment, the apparatus also includes a haptic element operationally coupled with the indicator configured to indicate the targeting accuracy.

In one embodiment, the apparatus also includes a display unit operationally coupled with the indicator configured to indicate the targeting accuracy, where a shape, a size or a color of the image indicates the targeting accuracy.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIG. 1 is a partially schematic view of an ultrasound system in accordance with conventional technology;

FIG. 2 is a cross-sectional view of an ultrasound probe in accordance with conventional technology;

FIG. 3 is an isometric view of an ultrasound probe in operation in accordance with conventional technology;

FIGS. 4A and 4B are bottom views of ultrasound probes in accordance with embodiments of the present technology;

FIG. 4C is a partially schematic isometric view of an ultrasound probe in accordance with an embodiment of the present technology;

FIG. 5 is a flow diagram of a method of operating an ultrasound transducer in accordance with an embodiment of the present technology;

FIG. 6 is an isometric view of an ultrasound probe in operation in accordance with an embodiment of the present technology;

FIG. 7 is an isometric view of an ultrasound probe in operation in accordance with an embodiment of the present technology;

FIGS. 8A and 8B are bottom and top views, respectively, of an ultrasound probe in accordance with an embodiment of the present technology;

FIGS. 9A and 9B are side views of ultrasound probes in accordance with an embodiment of the present technology;

FIG. 10 is a spectral graph of a sound waveform in accordance with an embodiment of the present technology;

FIG. 11A is a graph of ultrasound bursts in accordance with an embodiment of the present technology; and

FIG. 11B is a spectral graph of a sound waveform in accordance with an embodiment of the present technology.

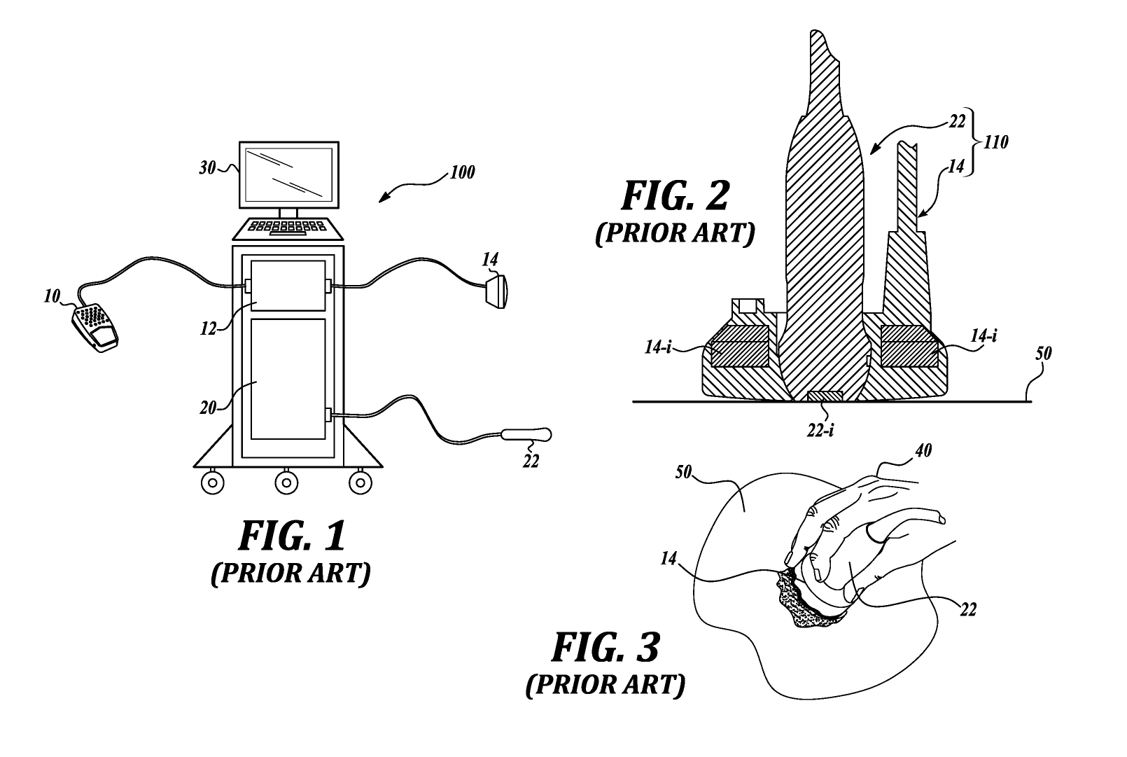

### DETAILED DESCRIPTION

While several embodiments have been illustrated and described, it will be appreciated that various changes can be made therein without departing from the spirit and scope of the inventive technology.

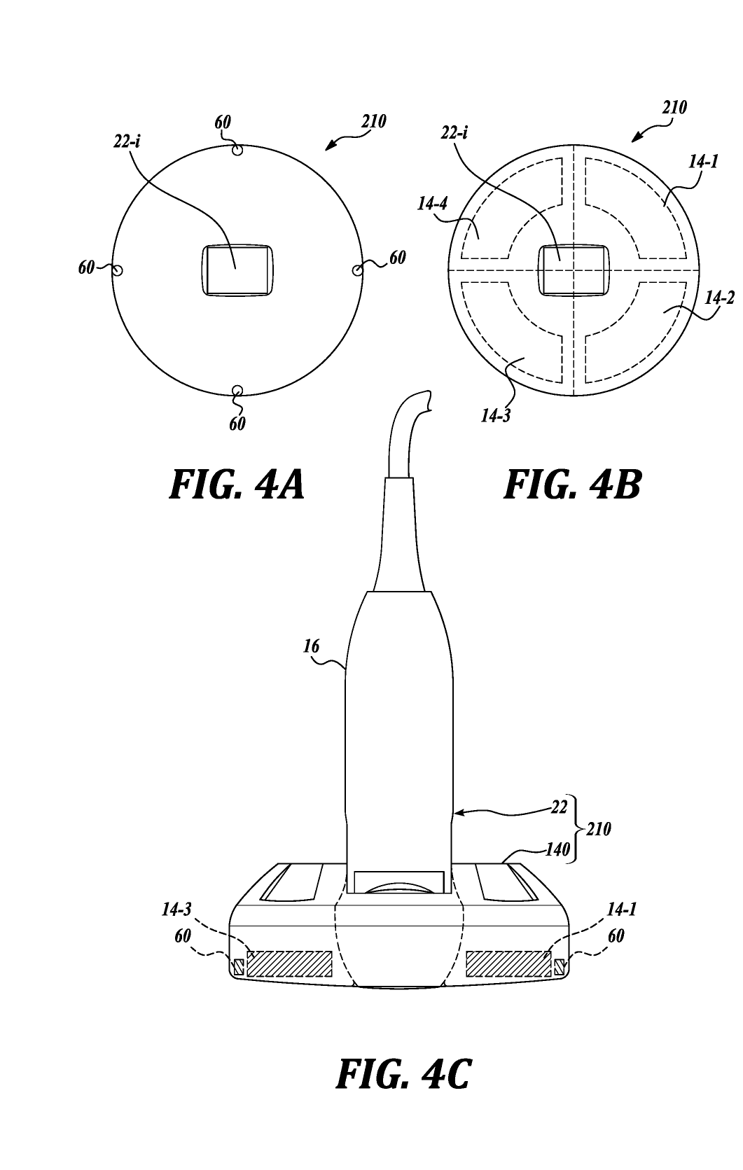

FIGS. 4A and 4B are bottom views of ultrasound probes 210 in accordance with embodiments of the present technology. FIG. 4A shows the ultrasound probe 210 that includes multiple microphones 60 that respond to the sound emissions caused or induced by the interaction of therapeutic ultrasound with the target object. In operation, the frequency of transmitted therapy ultrasound may be different from the sound arising from, for example, nonlinear interactions between the therapy ultrasound and the object of interest, such that the sound emission (also referred to as the sound waveforms) falls within the sensitivity range of the microphones 60, even though the frequency of the therapy ultrasound is outside of the audible range. In some embodiments, determination of the location of the object of interest may be improved by analyzing time delays among the signals received by different microphones 60, differences in the intensities of the signals, differences in the phases of the signals, etc., in order to determine direction-toward and/or distance-from the object of interest (e.g., a calcification or a bodily stone).

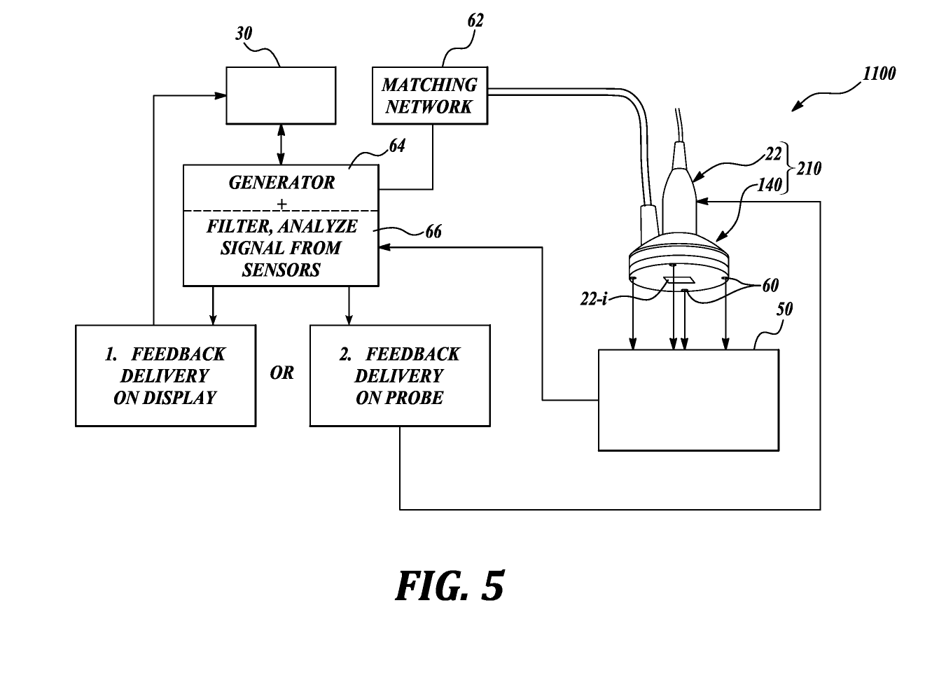

FIG. 4B shows the ultrasound probe 210 that includes multiple piezoelectric elements 14-i of the therapy transducer 140. Collectively, the piezoelectric elements 14-i operate as a phased array therapy transducer. In some embodiments, in addition to transmitting the therapy ultrasound toward the target, the elements 14-i of the phased array transducer may also receive and register the sound emissions induced by the interaction of therapeutic ultrasound with the target object. Some examples of such elements 14-i are the piezoelectric elements that vibrate when submitted to the AC current, but also generate AC current when subjected to the vibrations caused by the sound emissions associated with the target object. In operation, the location of the object of interest may be determined by analyzing, for example, time delays among the signals received by different elements 14-i, differences in the intensities of the signals, differences in the phases of the signals, etc., as explained in more detail with respect to FIG. 5 below.

The illustrated ultrasound probes 210 in FIGS. 4A and 4B include the imaging probe 22 having an imaging transducer 22-i. However, in different embodiments, the ultrasound probe 210 may not include the imaging probe 22 by relying exclusively on, for example, other sensing elements like the microphones 60 or the elements 14-i of the phased array transducer. The imaging ultrasound waveform may include a pulsed-wave Doppler (PWD) ultrasound.

FIG. 4C is a partially schematic isometric view of an ultrasound transducer 210 in accordance with an embodiment of the present technology. The illustrated ultrasound transducer includes the microphones 60 and the elements 14-i of the phased array therapy transducer 140. In different embodiments, the microphones 60 and the elements 14-i may operate separately or collectively to determine location of the object of interest. The imaging probe 22 and the therapy probe 140 may be combined as detachable units of the ultrasound probe 210. In some embodiments, the imaging probe 22 and the therapy probe 140 may be carried by a single housing.

FIG. 5 is a flow diagram of a method 1100 of operating an ultrasound transducer in accordance with an embodiment of the present technology. In operation, the therapy transducer 140 transmits therapy ultrasound toward the patient 50. The sound emissions (also referred to as the sound waveforms) caused by interactions between the therapy ultrasound and the target object may be detected by the microphones 60 and/or by the elements of the therapy transducer 140.

In different embodiments, different mechanisms may cause generation of sound arising from the interaction of the therapy ultrasound with the object (e.g., a bodily stone). In general, these mechanisms are nonlinear, and a consequence of nonlinearity is that the emitted sound frequency may be dramatically different from the frequency of the incident therapy ultrasound. For example, therapy ultrasound at a frequency of hundreds of kHz can give rise to audio and sub-audio frequencies in the range from 10 Hz (or lower) to tens of kHz (and much higher, as well). In contrast, a linear effect such as reflection of the therapy ultrasound from the stone preserves the frequency of the incident therapy ultrasound, such that a therapy ultrasound wave at hundreds of kHz gives rise to a reflected ultrasound wave at the same frequency. Some examples of the mechanisms that generate sound based on interactions between the therapy ultrasound (or diagnostic ultrasound) and the targeted object are discussed below.

1. Cavitation Bubble Collapse

Cavitation bubbles form in the presence of large negative pressure during the negative half-cycle of a therapy ultrasound waveform. Cavitation bubbles form more easily when there are cavitation nuclei present. Cavitation nuclei can consist of tiny dust particles (e.g., stone fragments), small features on the surface of a large stone (e.g., a micro-crack), small bubbles attached to the stone or hiding in a micro-crack, or free-floating micro-bubbles. Once the high pressure sound waves initiate cavitation (forming a small bubble), the bubble begins to oscillate in size from larger during the negative pressure half-cycle (rarefaction) to smaller during the positive half-cycle (compression). During this oscillation process, the bubble may grow in size from cycle to cycle through the process of rectified diffusion, and under certain circumstances, a cavitation bubbles may collapse violently, emitting a loud impulse of sound that can be captured by, for example, microphones 60.

The intensity of the sound is dependent on the number of cavitation events, which is affected by the concentration of cavitation nuclei, which is typically much greater at the stone surface or in the immediate vicinity of the stone compared to regions of soft tissue or fluid. In addition, when a stone fractures, it often releases a cloud of microscopic debris and/or micro-bubbles that briefly increases the concentration of cavitation nuclei in the vicinity of the stone, temporarily increasing the intensity of the sound. Through this mechanism, the location of a stone and the rate/effectiveness of the stone breaking may be identified by the intensity of these sounds.

2. Stone Fracture

The therapy ultrasound can promote stone fracture through several mechanisms. For example, localized stress concentrations arising from sound waves propagating through the bulk or over the surface of the stone can exceed the fracture strength of the stone material, causing a localized fracture. In addition, these stress concentrations can cause the growth of existing micro-cracks in the stone that were present from the outset or that arise from other mechanism. One potential source for initiating micro-cracks in the stone surface is the damage caused by cavitation bubble collapse. Thus, a cavitation nucleus present on the stone surface may give rise to a cavitation bubble which may then collapse violently, inducing damage (e.g., a micro-crack) on the stone surface. Through the process of crack formation and growth, there may be instances where a crack rapidly propagates, releasing broad band sound (e.g., a crack or pop sound). This broad band sound may be captured by, for example, microphones 60 or by the transducers 14-i of the therapy probe 140.

3. Radiation Pressure

When sound waves are absorbed or reflected from an object, they impart momentum to the object, which can be interpreted as a pushing force or pressure on the object, trying to push the object in the direction of sound propagation. In the typical application of burst-wave lithotripsy, the therapy ultrasound is characterized by brief (e.g., 100 microsecond) bursts of high intensity ultrasound repeated at a relatively low burst repetition frequency (e.g., 10 to 100 Hz). Inside the body, the stone may be constrained by the surrounding tissue from gross movement, but the intermittent pushes induced by radiation pressure may cause the stone to move back and forth at the burst repetition frequency (or a harmonic frequency thereof). In some embodiments, the sound associated with this vibration may not be strong enough to be easily detected by a microphone, but the vibratory motion may be detected and translated into an audio signal using, for example, the pulsed-wave Doppler ultrasound of the ultrasound imaging probe 22.

4. Nonlinear Propagation

Nonlinear propagation of high-pressure therapy ultrasound through fluid or body tissue may affect the frequency content of the waves, but the nonlinear propagation may also add second harmonic content (and higher harmonics). Furthermore, the waveform distortion caused by nonlinear propagation may affect the cavitation process itself

5. Frequency Content of the Transmitted Ultrasound Pulse

The sound may arise based on using a pulse instead of a continuous-wave exposure. A pulse contains not just a single frequency component, but a spectrum of frequencies, including those in the audible range. The strength of the sound components depends on the envelope of the waveform. Stated differently, the linear ultrasound pulse may already contain the audible noise. Furthermore, the sound acquired by, for example, microphones 60, may have different amplitudes due to a difference in scattering (e.g., caused by change in cavitation or by stone fragmentation).

Continuing with the discussion of FIG. 5, the acquired sound signal may be passed through a matching network 62 onto an analyzer 66 that filters and analyzes the acquired signals. Some nonexclusive examples of such analyzers 66 are frequency filters, phase filters, spectrum analyzers, computer routines (e.g., Matlab functions) that process phase delays or that determine location of the target object based on the acquired signals, etc.

In some laboratory experiments and in in-vivo pre-clinical porcine trials, the illustrated methods produced distinct audible or haptic feedback signals used for improved targeting of the target object (e.g., a kidney stone). In particular, the amplitude of the feedback signal (e.g., the amplitude of the sound emission associated with the target object) is significantly higher when the target object is within the focal zone of the ultrasound therapy probe 140. In some embodiments, a strong feedback signal was produced at the 10 Hz pulse repetition rate (burst repetition rate) of the ultrasound therapy probe 140.

In other laboratory experiments, it was observed that strong cavitation at the target object may increase the amplitude of the reflected therapy ultrasound, reflected imaging ultrasound, or the sound emission associated with the target object (e.g., caused by the therapy ultrasound interacting with the target object). Since, in some embodiments, strong cavitation is, in fact, undesirable, the method 1100 may guide the operator to target the ultrasound therapy probe 140 away from the zone of cavitation if the reflected therapy ultrasound exceeds a predetermined threshold.

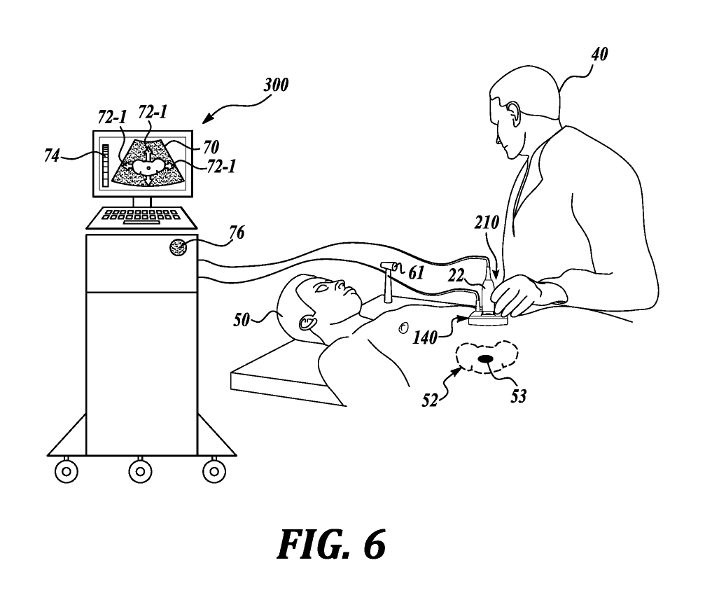

A generator 64 (e.g., a function generator or a source of electrical signals) may generate targeting signals related to the location of the object of interest and/or accuracy of the targeting of the object of interest. In some embodiments, targeting signals include location and/or shape of the target object shown on the display 30. The determination of the location/size/shape of the target object may also rely, at least partially, on the images obtained by the imaging transducer 22. In some embodiments, targeting signals may be displayed or exhibited on the therapy transducer 140 itself, or otherwise proximately to the operator. In different embodiments, targeting signals may be visual, haptic, audible, etc., as explained in more detail with FIGS. 6-9B below.

FIG. 6 is an isometric view of an ultrasound probe in operation in accordance with an embodiment of the present technology. In some embodiments, an operator 40 aims the ultrasound probe 210 at a target object 53 (e.g., a bodily stone) within a target area 52. The sound emissions associated with the target object may be detected by a bedside microphone 61, the elements of the ultrasound therapy transducer 140, and/or elements of the ultrasound imaging probe 22. The reflected signals may be processed using, for example, methods discussed with reference to FIG. 5. In some embodiments, an image 70 of the target object 53 may be shown on a display 300 together with targeting indicators. Some nonexclusive examples of such targeting indicators are arrows 72, targeting icons 74 (e.g., a thermometer, a crosshair, etc.), targeting oval, pulsating icons, and color-changing icons (indicating that the targeting is “hot” or “cold”). In some embodiments, an audible speaker 76 may emit targeting sounds (e.g., a clicking sound) and/or targeting instructions. In operation, the operator 40 can adjust the targeting of the target object 53 based on these targeting indicators.

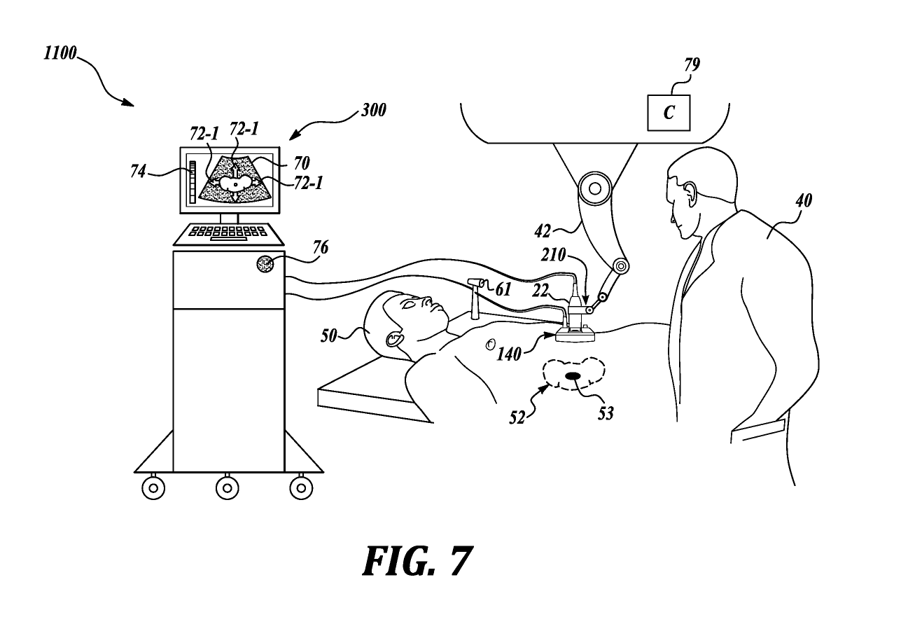

FIG. 7 is an isometric view of an ultrasound probe in operation in accordance with an embodiment of the present technology. With the illustrated embodiment, the ultrasound probe 210 is attached to a robotic manipulator 42. The targeting indicators may be provided to the robotic manipulator 42 through, for example, a controller 79. In operation, the robotic manipulator 42 interprets the targeting indicators and aims the ultrasound probe 210 toward the target object of interest. In different embodiments, the operator 40 may at least partially direct the operation of the robotic manipulator 42.

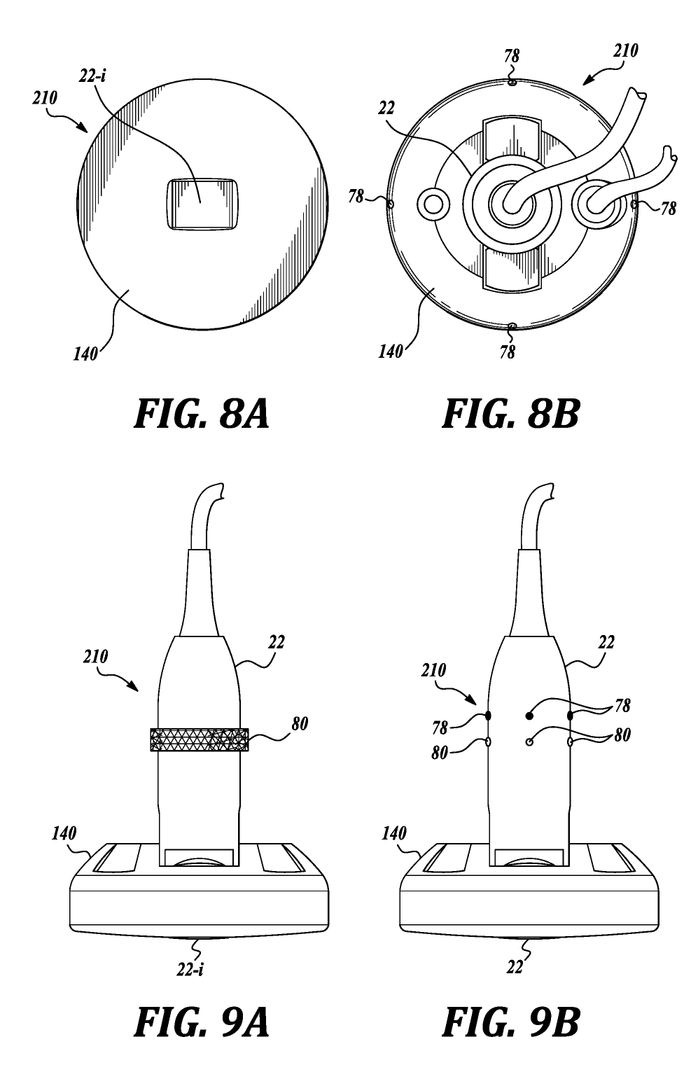

FIGS. 8A and 8B are bottom and top views, respectively, of an ultrasound probe 210 in accordance with an embodiment of the present technology. The illustrated ultrasound probe 210 in FIG. 8A includes the imaging transducer 22. However, in different embodiments the ultrasound probe 210 without the imaging transducer may also be used.

FIG. 8B shows the top view of the ultrasound probe 210. The illustrated ultrasound probe 210 includes visual indicators 78. In different embodiments, the visual indicators 78 may be, for example, light emitting diodes (LED), blinking lights, small lightbulbs, termination points of fiber optics, etc. In operation, the visual indicator 78 provides targeting indicators for the operator. Such targeting indicators may be the intensity of the light, direction of the light, frequency of blinking of the light, color of the light (e.g., color of the LED), etc. Based on these targeting indicators, the operator may improve the targeting of the ultrasound probe 210 toward the object of interest. FIGS. 9A and 9B are side views of an ultrasound probe 210 in accordance with an embodiment of the present technology. FIG. 9A shows the ultrasound probe 210 with a haptic indicator 80. In operation, the operator holds the ultrasound probe 210 at least partially by the haptic indicators 80. The haptic indicator 80 provides targeting instructions or clues to the operator by, for example, providing stronger haptic indications in the direction in which the operator should move the ultrasound probe 210 for an improved targeting. FIG. 9B shows the ultrasound probe 210 with distributed haptic indicators 80 and the visual indicators 78. In some embodiments, the operator may rely on a combination of the haptic indicators 80 and visual indicators 78 to improve the targeting of the ultrasound probe 210.

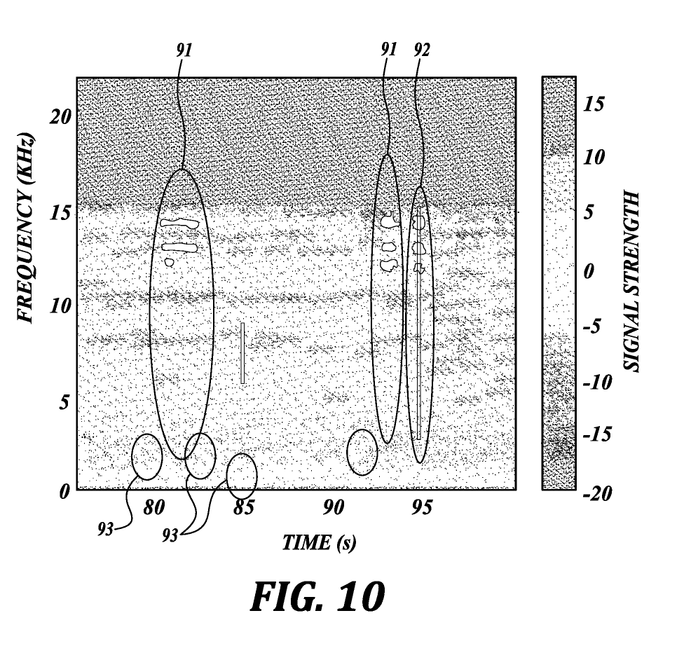

FIG. 10 is a spectral graph of the sound emissions associated with the target object in accordance with an embodiment of the present technology. The horizontal axis represents time in seconds, and the vertical axis represents frequency in kHz. The shades in the graph represent spectral content of the signal, which is a proxy for the signal strength of the sound emissions (on a logarithmic scale). Circled areas 91 correspond to a relatively high amplitude, but a longer duration signal (lasting several seconds), indicating generally successful targeting of the calcification in the body. A circled area 92 corresponds to a narrower signal, lasting about one second or less, but having higher amplitude (comparable to a loud click when something is being hit). In operation, such signals 91 and/or 92 may provide targeting guidance to the operator. Circled areas 93 at the lower frequencies (about 3 kHz) correspond to the voices of people in the room.

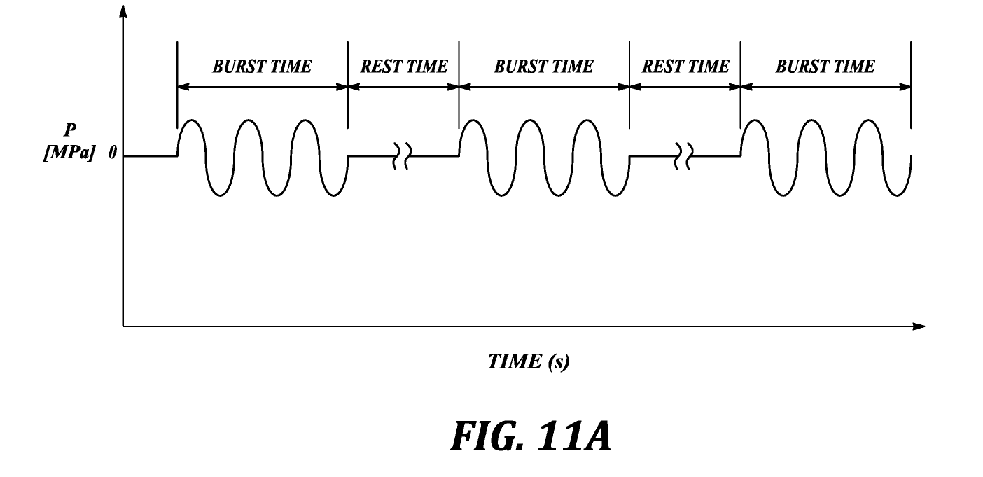

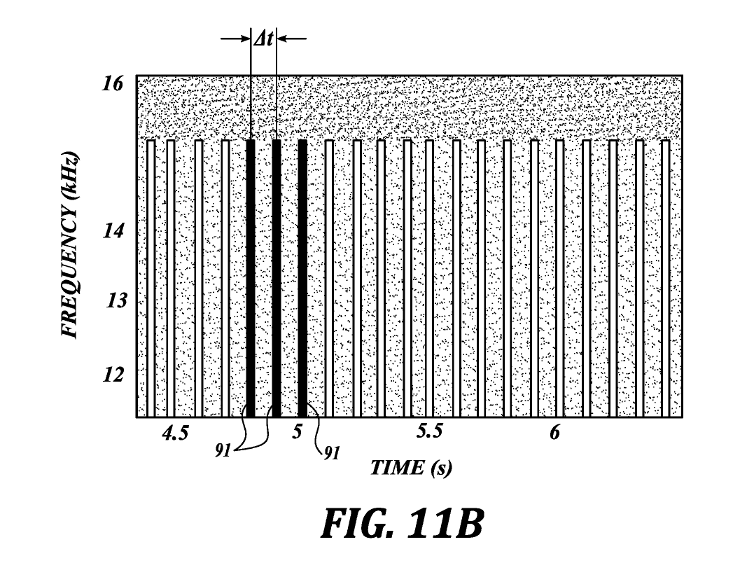

FIG. 11A is a graph of ultrasound bursts in accordance with an embodiment of the present technology. The horizontal axis represents time in seconds, and the vertical axis represents pressure of the ultrasound waves in MPa. The illustrated bursts corresponding to the “burst time” include smooth ultrasound waves. These bursts of the BWL are separated by the rest times. Therefore, the illustrated BWL waveforms include at least 2 frequencies: the frequency of the ultrasound waves within the bursts, and the frequency of the repetition of the bursts. Generally, the frequency of the bursts may be significantly smaller than the frequency of the ultrasound waves within the individual bursts. For example, is some embodiments, the frequency of the bursts may be within the audible range of frequencies. In some embodiments, signals at one or both of these frequencies may reflect off the target object as a reflected frequency that is detectable by the microphones 60 or the transducer elements 14-i of the phased array therapy transducer. In some embodiments, one or both of these frequencies undergoes nonlinear interactions resulting in additional frequencies being detectable by the microphones 60 or the transducer elements 14-i of the phased array therapy transducer. These additional frequencies may improve sensitivity and usefulness of the inventive technology. An example of the spectral graph obtained by emitting a BWL therapy ultrasound and acquiring the sound waveforms recorded by a microphone is discussed below with reference to FIG. 11 B.

FIG. 11B is a spectral graph of a sound waveform in accordance with an embodiment of the present technology. The horizontal axis represents time in seconds, and the vertical axis represents frequency in Hertz. The shades in the graph represent spectral density of the signal, which is a proxy for the signal strength of the reflected therapy ultrasound. The period Δt between the adjacent spikes in the signal strength corresponds to the repetition period of the BWL bursts. In the illustrated example, this period of about 0.1 seconds corresponds to a burst repetition frequency of about 10 Hz. In other words, the amplitude of sound waveform is modulated at the frequency of the BWL bursts. The shaded areas 91 having higher amplitude in the 11.5 kHz to 15 kHz frequency band correspond to successful targeting of the target object by the therapy ultrasound of the BWL. Therefore, in some embodiments of the inventive technology, the successful targeting of the object may be detectable by analyzing the strength of the received audio band signal coinciding with the therapy ultrasound bursts. For example, the operator may conclude that the targeting was relatively successful at the three consecutive times corresponding to the shaded areas 91.

Many embodiments of the technology described above may take the form of computer- or controller-executable instructions, including routines executed by a programmable computer or controller. Those skilled in the art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

I/We claim:

1. A method for targeting an object in a body using ultrasound, comprising:
producing a therapy ultrasound waveform configured to fragment or comminute the object in the body using a therapy transducer of an ultrasound probe;
acquiring a sound waveform by a receiver, wherein the sound waveform is at least in part caused by interactions of the therapy ultrasound with the object; and
generating an indication of a targeting accuracy based on the acquired sound waveform.

2. The method of claim 1, wherein the object is a stone or a calcification.

3. The method of claim 1, wherein the receiver comprises a microphone.

4. The method of claim 3, wherein the microphone and the therapy transducer are carried by a common housing of the ultrasound probe.

5. The method of claim 3, wherein the microphone and the therapy transducer are separate.

6. The method of claim 3, further comprising:
converting a microphone signal into a digitized signal in a time domain;
processing the digitized signal into a frequency spectrum;
detecting at least one extremum in the frequency spectrum; and
determining the targeting accuracy based on the at least one extremum of the frequency spectrum.

7. The method of claim 1, wherein generating the indication of the targeting accuracy comprises generating an audible feedback or a light feedback.

8. The method of claim 1, wherein generating the indication of the targeting accuracy comprises generating a haptic feedback.

9. The method of claim 1, wherein generating the indication of the targeting accuracy comprises generating an image on a display unit, wherein a shape, a size or a color of the image indicates the targeting accuracy.

10. The method of claim 1, further comprising:
retargeting the therapy ultrasound waveform based on the indication of the targeting accuracy.

11. The method of claim 10, wherein the retargeting the therapy ultrasound waveform comprises robotically retargeting the therapy ultrasound waveform.

12. The method of claim 10, wherein the therapy transducer is a phased array therapy transducer comprising a plurality of individually operable transducer elements, the method further comprising:
retargeting the therapy ultrasound waveform by controlling individual transducer elements of the phased array.

13. The method of claim 1, wherein the sound waveform comprises sound emissions from cavitation bubbles generated by the therapy ultrasound waveform.

14. The method of claim 1, wherein the therapy ultrasound waveform is transmitted in bursts, wherein a frequency of the bursts is within an audible range of frequencies, and wherein a modulation frequency of the sound waveform coincides with the frequency of the bursts.

15. The method of claim 1, further comprising:
generating a Doppler ultrasound audio waveform using an imaging transducer of the ultrasound probe; and
generating a display representative of the object motion based on the Doppler ultrasound audio waveform.

16. The method of claim 15, wherein the imaging ultrasound waveform comprises a pulse wave Doppler (PWD) ultrasound.

17. An apparatus for treating an object in a body using ultrasound, comprising:
an ultrasound probe comprising:
a therapy transducer configured to fragment or comminute the object in the body by a therapy ultrasound, and
an imaging probe configured to image the object by an imaging ultrasound;
a receiver configured to detect a sound waveform, wherein the sound waveform is at least in part caused by interactions of the therapy ultrasound with the object; and
an indicator configured to indicate a targeting accuracy based on the sound waveform detected by the receiver.

18. The apparatus of claim 17, wherein the object is a stone or a calcification.

19. The apparatus of claim 17, wherein the receiver is a microphone.

20. The apparatus of claim 17, further comprising a controller configured to adjust a target therapy zone based on the targeting accuracy.

21. The apparatus of claim 20, further comprising a robotic arm configured to adjust a position of the ultrasound probe.

22. The apparatus of claim 20, wherein the therapy transducer is a phased array therapy transducer comprising a plurality of individually operable transducer elements, and wherein the controller is configured to adjust the target therapy zone by controlling individual transducer elements of the phased array.

23. The apparatus of claim 17, wherein the therapy transducer is a phased array therapy transducer comprising a plurality of individually operable transducer elements that are configured to generate the therapy ultrasound and to detect the sound waveform.

24. The apparatus of claim 17, wherein the receiver comprises a bed-side microphone.

25. The apparatus of claim 17, wherein the receiver comprises a microphone, and wherein the microphone and the therapy transducer are carried by a common housing of the ultrasound probe.

26. The apparatus of claim 17, further comprising at least one of a speaker or a source of light operationally coupled with the indicator configured to indicate the targeting accuracy.

27. The apparatus of claim 17, further comprising a haptic element operationally coupled with the indicator configured to indicate the targeting accuracy.

28. The apparatus of claim 17, further comprising a display unit operationally coupled with the indicator configured to indicate the targeting accuracy, wherein a shape, a size or a color of the image indicates the targeting accuracy.
